(19) United States
(12) Patent Application Publication
(10) Pub. No.: US 2019/0355478 A1
(43) Pub. Date: Nov. 21, 2019

(54) SYSTEMS AND METHODS FOR AN EXPERT SYSTEM FOR PRECISION ONCOLOGY

(71) Applicant: PERTHERA, INC., McLean, VA (US)

(72) Inventors: Subha MADHAVAN, Potomac, MD (US); Michael Jon PISHVAIAN, Potomac, MD (US); Robert Joseph BENDER, Rockville, MD (US); Edik Matthew BLAIS, Charlottesville, VA (US); Craig HEARTWELL, Herndon, VA (US); Samuel Brewster LESLIE, Burke, VA (US); David Charles HALVERSON, North Bethesda, MD (US); Jonathan Robert BRODY, Philadelphia, PA (US); Emanuel Frank PETRICOIN, III, Gainesville, VA (US)

(73) Assignee: PERTHERA, INC., McLean, VA (US)

(21) Appl. No.: 16/475,349

(22) PCT Filed: Dec. 29, 2017
(86) PCT No.: PCT/US17/68953

§ 371 Date: Jul. 1, 2019

Related U.S. Application Data
(60) Provisional application No. 62/440,781, filed on Dec. 30, 2016.

(51) Int. Cl. G16H 50/70; A61B 5/00; A61N 5/10

(52) U.S. Cl. CPC G16H 50/70; A61B 5/7275; G16H 20/40; A61N 2005/1041; A61N 5/103

(57) ABSTRACT

A method and system for generating a therapy plan for a patient is described. The method includes providing a knowledgebase of annotated disease treatment information, a patient history database including disease types for a plurality of patients and a molecular database including molecular data associated with patients, selecting at least one of the plurality of patients for analysis, identifying a list of one or more candidate therapy plans from the knowledgebase based on at least one of the disease type and the molecular data for the at least one patient, and scoring each of the one or more candidate therapy plans by combining a molecular vector, a disease-specific vector and a patient history vector. The therapy plan with the highest score can then be selected for implementation. The system includes various hardware components for implementing the various functions as defined by the method for generating the treatment plan.

### CROSS-REFERENCE TO RELATED APPLICATIONS

This application claims benefit of and priority to U.S. Provisional Patent Application No. 62/440,781 entitled “Systems and Methods for an Expert System for Precision Oncology,” filed Dec. 30, 2016, the disclosure of which is incorporated by reference herein in its entirety.

### TECHNICAL FIELD

The present disclosure relates generally to expert systems and, more particularly, to systems and methods for annotating scientific research, receiving patient medical history information and applying filtering, rules and scoring to rank treatments and finalize a set of treatments with expert input. The present disclosure also relates to generating therapy plans for a patient and scoring them.

### BACKGROUND

Genetic information from patients allows the possibility of precision oncology and treatments for patients that are individualized. However, genetic information alone may not be sufficient. For example, a genomic profiling study reveals actionable mutations affecting signaling pathways, but in spite of these mutations, targeted inhibitors of these pathways may have low success rates. A possible reason for these failures is that single-gene biomarkers may fail to account for crosstalk within and between dysregulated pathways. Multi-omic profiling based on multiple biomarkers, genetic and molecular information, and patient history can help make better molecular recommendations for treatment.

There has been an explosion in the number of drugs being developed specifically for cancer—nearly 1,000 of them are now at various stages of being tested for safety and efficacy. This growth in new drugs is associated with an evolution of precision medicine. However, it is unlikely that an oncologist or an entire oncology team treating a particular patient can keep up with all the science and progress being established by these clinical trials or keep up with all of the published literature on disease treatments.

Accordingly, there remains a need for a system that accesses and organizes scientific knowledge and studies on a real-time basis. There remains a further need for a computational framework for integrating a knowledgebase with multi-omic data and drug response data from cancer cell lines to propose “actionable” biomarkers based on a panel of pathways. There is a further need to identify and rank drugs or treatments that may be applicable to patients based on patient specific information and scientific knowledge to provide complete information necessary for a patient and oncologist to discuss treatment options.

### SUMMARY

A method of generating a therapy plan for a patient is described. The method includes providing a knowledgebase of annotated disease treatment information, a patient history database including disease types for a plurality of patients and a molecular database including molecular data associated with patients; selecting at least one of the plurality of patients for analysis; identifying a list of one or more candidate therapy plans from the knowledgebase based on at least one of the disease type and the molecular data for the at least one patient; and scoring each of the one or more candidate therapy plans by combining a molecular vector, a disease-specific vector and a patient history vector.

In some implementations of the above-identified method, the molecular database can include information concerning genomic and protein profiles of the at least one patient, the knowledgebase can include information concerning a disease in the at least one patient, and the patient history database can include information concerning a prior therapy history of the at least one patient.

In some implementations of the above-identified method, the molecular vector can be determined according to scoring rules based on the molecular database, the disease-specific vector can be determined according to scoring rules based on the knowledgebase, and the patient history vector can be determined according to scoring rules based on the patient history database.

In some implementations of the above-identified method, the identifying the list can include filtering initial therapy plans based on the patient history database.

In some implementations of the above-identified method, the identifying the list can include identifying a disease or a state of disease progression in the at least one patient based on the molecular database, the knowledgebase, and the patient history database.

In some implementations of the above-identified method, the identifying the list can include receiving at least one of (i) a query result from the molecular database concerning the molecular data of the at least one patient, (ii) a query result from the knowledgebase concerning the disease type of the at least one patient, and (iii) a query result from the patient history database concerning a prior therapy history of the at least one patient.

In some implementations of the above-identified method, the scoring can include ranking the list of one of more candidate therapy plans.

In some implementations of the above-identified method, the therapy plan can include a pharmaceutical treatment plan with one or more pharmaceutical drugs.

In some implementations of the above-identified method, the at least one patient is a cancer patient.

In some implementations of the above-identified method, the at least one patient is a pancreatic cancer patient.

In some implementations of the above-identified method, the molecular data can include genomic and protein profiles of the at least one patient.

A method of treating cancer is also described. The method of treating cancer includes generating a therapy plan for a cancer patient according to the method of any one of the proceeding claims, and administering one or more pharmaceutical drug in accordance with the therapy plan.

A system for generating a therapy plan for a patient is also described. The system includes a knowledgebase of annotated disease treatment information, a patient history database including disease types for a plurality of patients, and a molecular database including molecular data associated with patients; a memory including program instructions for assembling molecular, disease-specific and patient history vectors from the databases, and filtering and scoring at least one candidate therapy for at least one of the plurality of patients; and a processor coupled to the databases and the memory. The processor may be capable of executing the program instructions to identify a list of one or more candidate therapy plans from the knowledgebase based on at least one of the disease type and the molecular data for the at least one patient; compile a molecular vector, a disease-specific vector and a patient history vector, and score each of the at least one candidate therapy plans for each of the at least one patient based on the molecular vector, the disease-specific vector and the patient history vector.

In some implementations of the above-identified system, the molecular database can include information concerning genomic and protein profiles of the at least one patient, the knowledgebase comprises information concerning a disease in the at least one patient, and the patient history database comprises information concerning a prior therapy history of the at least one patient.

In some implementations of the above-identified system, the molecular vector can be determined according to scoring rules based on the molecular database, the disease-specific vector is determined according to scoring rules based on the knowledgebase, and the patient history vector is determined according to scoring rules based on the patient history database.

In some implementations of the above-identified system, the identifying the list can include filtering initial therapy plans based on the patient history database.

In some implementations of the above-identified system, the identifying the list can include identifying a disease or a state of disease progression in the at least one patient based on the molecular database, the knowledgebase, and the patient history database.

In some implementations of the above-identified system, the identifying the list can include receiving at least one of (i) a query result from the molecular database concerning the molecular data of the at least one patient, (ii) a query result from the knowledgebase concerning in the disease type of the at least one patient, and (iii) a query result from the patient history database concerning a prior therapy history of the at least one patient.

In some implementations of the above-identified system, the scoring can include ranking the list of one of more candidate therapy plans.

In some implementations of the above-identified system, the therapy plan can include a pharmaceutical treatment plan with one or more pharmaceutical drugs.

In some implementations of the above-identified system, the at least one patient is a cancer patient.

In some implementations of the above-identified system, the at least one patient is a pancreatic cancer patient.

In some implementations of the above-identified system, the molecular data can include genomic and protein profiles of the at least one patient.

### BRIEF DESCRIPTION OF THE DRAWINGS

The accompanying drawings, which are incorporated in and form a part of the specification, illustrate the embodiments of the present disclosure and together with the written description serve to explain the principles, characteristics, and features of the disclosure. In the drawings:

FIG. 1 depicts a sample system for treating disease in accordance with an embodiment.

FIG. 2 depicts a process for collecting an annotating literature and creating molecular and patient history databases in accordance with an embodiment.

FIG. 3 depicts a process for analyzing a patient disease and potential treatments based on patient history, molecular data, and annotated literature to produce a report in accordance with an embodiment.

FIG. 4 illustrates a set of sample tables that may be stored in one or more associated databases in accordance with an embodiment.

FIG. 5 depicts scoring techniques in accordance with an embodiment.

FIG. 6 depicts an OnBoard process for facilitating expert and physician evaluation of treatment considerations and expert review of report information associated with patients and potential treatments in accordance with an embodiment.

FIG. 7 depicts an illustrative view of molecular, disease specific and patient history vectors in accordance with an embodiment.

FIG. 8 depicts a computer system for use with the system for treating disease as described herein.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The embodiments of the present teachings described below are not intended to be exhaustive or to limit the teachings to the precise forms disclosed in the following detailed description. Rather, the embodiments are chosen and described so that others skilled in the art may appreciate and understand the principles and practices of the present teachings.

The present disclosure relates to using a sample drug scoring system for determining improved treatment regimens for a patient. The sample drug scoring system, such as that described herein, can use several (e.g., three) classes of information, referred to herein as vectors, to rank therapies according to their probability of improving patient outcomes. For example, a set of vectors can include a molecular vector, a disease-specific vector, and a patient history vector.

The molecular vector is adjusted based on the strength of the evidence supporting the relationship between a molecular profile and drug sensitivity. When the evidence supporting a biomarker is derived purely from pre-clinical experiments, the molecular score is low. Evidence from retrospective subgroup analysis of clinical trials results in a moderate score, and biomarker-stratified, prospective randomized clinical trials lead to a high score. Examples of biomarkers that typically carry low molecular scores are chemotherapy markers; many of these are based on cell line studies or retrospective analysis and are therefore given low scores. Examples of biomarkers with high molecular scores include oncogenic driver mutations such as EGFR mutations and AIK fusions, where the strength of evidence from clinical trials has led to FDA approval in tumors harboring the biomarker. In addition to the type of evidence supporting a biomarker, the aggregate effects of all alterations in the molecular profile are considered. A single driving molecular aberration is given a lower score than multiple alterations in the same signaling pathway. An example of a highly scored molecular profile could include multiple aberrations in the PI3K/mTOR pathway with PTEN loss, PIK3CA mutation and mTOR phosphorylation for recommendation of the mTOR inhibitor everolimus. When two or more molecular alterations present conflicting evidence, their impact on the molecular score can negate one another. Complex situations such as these are addressed by rules, which are defined using the interaction network in the knowledgebase (described below).

The disease-specific vector is based on the extent to which a single drug or combination of drugs has been tested in a particular disease type independent of any molecular alterations. Drug regimens with a clear lack of benefit are given low scores, while regimens with clinically proven benefit in the disease are given high scores. An example of a highly ranked regimen for a colorectal cancer patient might include FOLFOX+Avastin, which is FDA approved for this indication.

The patient treatment history vector captures the past exposure of the patient to the drugs in a combination, which often holds predictive value of the future response upon re-exposure. Low scores are assigned when a patient has received all agents with clear evidence of disease progression, whereas high scores are assigned when e patient is completely naïve to the regimen under consideration.

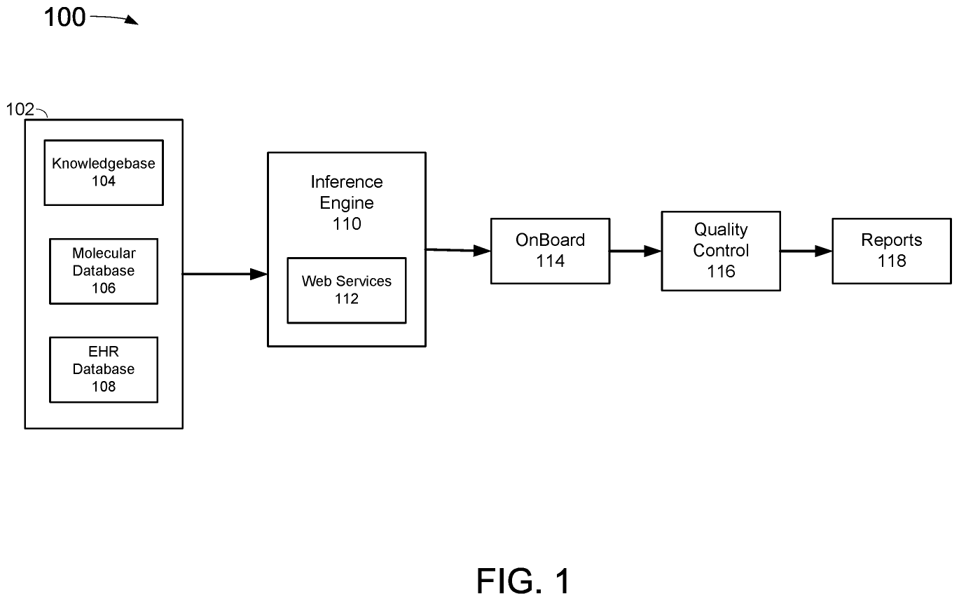

FIG. 1 depicts a system 100 according to a sample embodiment for recommending disease treatment for patients and doctors. Referring to FIG. 1, according to some embodiments of the invention, a set of databases 102 supporting a system for treatment of disease may include at least one knowledge database (“knowledgebase”) 104, at least one molecular database 106, and at least one electronic health record (“EHR”) or patient database 108. The databases 102 may be implemented and/or referred to as a single database or as multiple databases. It will be understood by one having ordinary skill in the art that the databases 102 may be one database or multiple databases and/or may be centralized or distributed. There may also be one schema or multiple schemas for accessing the databases. In some embodiments, the at least one knowledgebase 104, molecular database 106, and patient history database 108 may be combined in one or multiple databases.

An inference engine 110 may be coupled to the databases 102, typically over a network such as a LAN, WAN, or the Internet using one or more appropriate web services 112. However, it should be noted that the databases 102 could also be resident on a server that runs the inference engine 110 as well. The inference engine 110 may access the databases 102 using one or more queries and schemas. In some implementations, the queries and schemes may be configurable based upon the individual database structures. An OnBoard system 114 may be coupled to the inference engine 110 and the databases 102 in a similar manner (e.g., via a network). In some implementations, the OnBoard system 114 may be capable of implementing processes to facilitate expert review of disease treatments based on information or treatment recommendations output from the inference engine. For example, the OnBoard system 114 may output patient, treatment and/or disease information and recommendations to at least one quality control process 116. Based upon the information and recommendations, one or more reports 118 may be generated reflecting recommendation from the inference engine 110, an expert panel (e.g., provided via the OnBoard system 114), and a quality control process 116 that may be targeted to physicians, patients, epidemiologists, data feedback to the inference engine or other reports.

The following definitions are applicable to FIG. 1:

“Patient info” includes, but is not limited to, all clinically relevant patient data in regards to precision therapy, pathologic features, prior treatment, genetic mutations, etc.

“Molecular info” includes next generation sequencing (NGS), immune histo chemistry (IHC), and Phosphoprotein data from patient(s).

“Disease info” includes a primary diagnosis, approved standard of care (SOC) therapy for disease, experimental therapies for disease, and average outcome for disease.

Referring again to FIG. 1, the knowledgebase 104 may be implemented as a database that includes information about treatment of disease, such as cancer. It may be generic to a type of disease, such as cancer, or more specific, such as to one or more specific types of cancer. In some embodiments, the disease may be pancreatic cancer. Other disease types or subtypes may be the focus of the knowledgebase 104 in some embodiments. The knowledgebase 104 may be populated with information using previous experience with patients that have gone through the expert system, including patient outcomes, and information from medical literature and other cancer studies. The knowledgebase 104 becomes increasingly valuable as new information is added.

According to some embodiments, information in the knowledgebase 104 may include, but is not limited to, information on: medical trials, drugs, biomarker definitions, biomarker implications, and scientific literature. The information in the knowledgebase 104 may be extracted from scientific literature by an engine that accesses the scientific literature and filters it for new articles, studies or information that meet criteria, such as being published in a reputable or peer-reviewed journal or proceedings. Extracted information may be annotated and stored in the knowledgebase 104 by a server parsing studies received over a network or from a remote database. A record may be created in the database that is associated with the study. This process of creating records may be facilitated by a scientist interacting with the system, which accesses studies in real-time. Examples of information that may be associated with a study are shown below:

### Biomarker Definition

| | | |
|---|---|---|
| cohort_id | NSCLC | NSCLC |
| gene_symbol | EGFR | EGFR |
| assay_id | NGS | NGS |
| assay_lab | any | any |
| assay_result | Loss exon 19 | L858R |
| biomarker_id | EGFR{actionable} | EGFR{actionable} |
| biomarker_label | actionable | actionable |
| definition_description | clinically actionable | clinically actionable |
| | mutation associated | mutation associated |
| | with increased | with increased |
| | sensitivity to first | sensitivity to first |
| | generation EGFR | generation EGFR |
| | inhibitors in NSCLC | inhibitors in NSCLC |

### Biomarker Implication

| | | |
|---|---|---|
| cohort_id | any | any |
| assay_id | NGS | NGS |
| gene_symbol | EGFR | ATM |
| biomarker_label | L858R | Path |
| biomarker_id | EGFR{actionable} | ATM{Path} |
| Strategy_id | EGFRi | PARPi |
| Implication | EGFR actionable | ATM inactivating mutation |
| description | mutation was found. | was found. ATM encodes a |
| | This mutation in exon | kinase which plays a key role |
| | 21 is clinically known | in the response to DNA |
| | to confer sensitivity | double-stranded breaks, as |
| | to most EGFR | well as multiple other critical |
| | inhibitors and serves | cellular functions (PMID: |
| | as a clinically | 26777338). There are no |
| | actionable biomarker | approved therapies targeting |
| | in NSCLC. It is | ATM specifically, but cells |
| | important to note that | with ATM inactivation, like |
| | many clinical trials | other genomic instability |
| | may exclude patients | tumor suppressors, may have |
| | with this mutation | increased sensitivity to |
| | until they have | PARP inhibitors or platinum |
| | progressed on EGFR | agents (PMID: 21603316, |
| | inhibitor if indicated | PMID: 20739657) and |
| | as a standard option. | clinical trials including |
| | | PARP inhibitors could be |
| | | considered. |

### Drug Strategies

| strategy type | drug_id | strategy_id | strategy_name |
|---|---|---|---|
| targeted therapy | erlotinib | EGFRi | EGFR inhibitor |
| targeted therapy | gefitinib | EGFRi | EGFR inhibitor |
| targeted therapy | icotini | EGFRi | EGFR inhibitor |
| targeted therapy | lapatinib | EGFRi | EGFR inhibitor |

### Trials

| | | |
|---|---|---|
| trial_id | NCT02477644 | NCT02098343 |
| trial_title | Platine, Avastin and | p53 Suppressor Activation |
| | Olaparib in 1st Line | in Recurrent High Grade |
| | | Serous Ovarian Cancer, a |
| | | Phase Ib/II Study of |
| | | Systemic Carboplatin |
| | | Combination Chemotherapy |
| | | With or Without APR-246 |
| diagnosis_id | OV | OV |
| inclusion_subtype | endometrioid; | serous |
| | epithelial | |
| exclusion_subtype | borderline; | |
| | mucinous | |
| inclusion_criteria | IIIB; IV | LA; M |
| exclusion_criteria | met{CNS} | |
| trial_treatment | Olaparib | APR-246 + |
| | | Carboplatin/PLD |
| trial_comparator | Placebo | Carboplatin/PLD |
| inclusion_treatment | L1 | TL2; TL3; |
| | | platinum{sensitive} |
| exclusion_treatment | | |
| inclusion_biomarker | BRCA1{germline} \| | TP53{GOF} \| TP53{IHC |
| | BRCA2{germline} | Pos} |
| exclusion_biomarker | | TP53{LOF} |

According to some embodiments, the molecular database 106 may include information on molecular treatments. For example, the following molecular information may be accessed and stored in real time within the molecular database 106:

NGS—Next Generation Sequencing database, which includes information on 321 genes at the present time and may be accessed through FoundationOne.

Immuno Histo Chemistry database which includes information on 17 proteins.

PHO—Phosphoprotein data (signaling activated in cell cycle).

ISH—In situ hybridization (ALK, MET, ROS1, Her2)

According to some embodiments, the EHR database 108 may include medical history information for a plurality of patients. An EHR may include information such as past medical history, treatments, clinical file data, demographic information, general health information, treatment start and stop times, outcomes or responses to treatments, and patient location and contact information, among other information. According to some embodiments, the following information may illustratively be included in the EHR database 108:

| | | |
|---|---|---|
| PATIENT_NUMBER | pan-1032 | pan-1032 |
| TREATMENT_DRUG | Gemcitabine + nab- | 5-Fluorouracil + nal- |
| | Paclitaxel | Irinotecan |
| START_DATE | Jan. 19, 2016 | Apr. 25, 2016 |
| STOP_DATE | Mar. 29, 2016 | Oct. 18, 2016 |
| STOP_REASON | Progression | Ongoing |
| BEST_RESPONSE | PR | SD |
| TREATMENT_SETTING | 1st line | 2nd line |
| TREATMENT_CLASS | FDA Approved | FDA Approved |

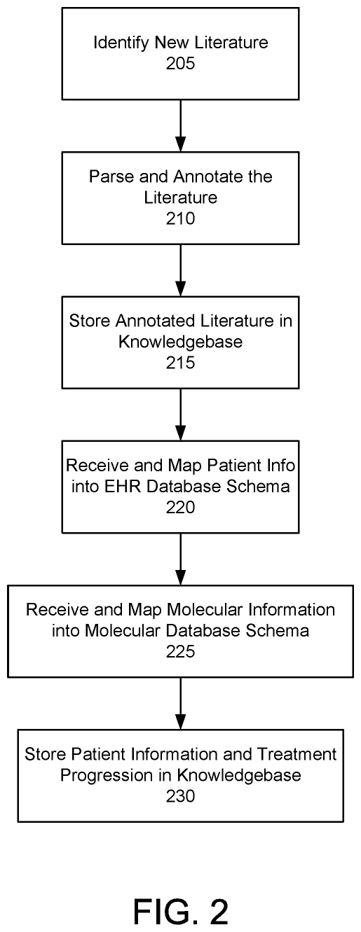

FIG. 2 depicts an illustrative process for obtaining information from literature, from patient medical histories, and molecular testing data and to map the information into one or more schemas and store the information into the databases 102 described above (e.g., the knowledgebase 104, the molecular database 106, and the EHR database 108 as appropriate) and make it available for querying. In general, the information is stored in fields that may be queried as described herein.

The process as shown in FIG. 2 may be performed by, for example, a computing device, such as a database administrator, configured to manage the databases 102. The process may include identifying 205 new literature related to one or more diseases included in the knowledgebase. The Literature may be parsed 210 and annotated, and the updated literature may be stored 215 in the knowledgebase. The database administrator may also receive 220 and map patient info into the EHR database schema. Similarly, the database administrator may receive 225 and map molecular information into the molecular database schema. The database administrator may further store 230 patient disease and treatment progression information in the knowledgebase for further analysis.

Referring again to FIG. 1, the inference engine 110 may be used to identify treatments based on information in the databases 102 associated with patients, molecules, and treatments. The inference engine 110 may query the databases 102 during the processing according to pre-determined database schemas that allows the inference engine to access the database(s) including the knowledgebase 104, the EHR database 108, and the molecular database 106.

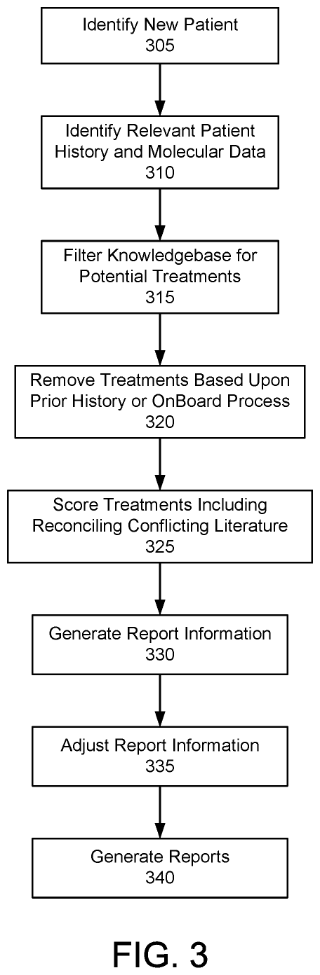

The inference engine 110 may be configured to perform the following functions, which are also shown as part of the analysis and report generating process in FIG. 3:

1. Identify key aspects of the patient's profile for identifying relevant treatments, including clinical information, such as the type of cancer, and prior treatment and molecular data. This is done by querying the EHR and molecular databases, for example. As shown in FIG. 3, this may include identifying 305 a new patient as well as identifying 310 relevant patient history and molecular data from the respective databases.

2. Query the knowledgebase to compile a list of relevant publications and standard of care options matching the disease type. As shown in FIG. 3, this may include filtering 315 the knowledgebase for potential treatments and related information.

3. Remove treatments that the patient has already received and developed resistance. As shown in FIG. 3, this may include removing 320 treatments based upon prior patient history information obtained during the OnBoard process (which is described in greater detail below).

4. Assign scores in three categories (molecular, disease, and patient) and an overall score based on the combination. As shown in FIG. 3, this may include scoring the individual treatments including reconciling 325 any conflicting literature filtered from the knowledgebase. Additionally, once the treatments are scored, the inference engine may generate 330 the report information, adjust 335 the report information based upon any feedback from the OnBoard process or quality control, and one or more reports may be generated 340.

An example of the inference engine is set forth below.

### Example 1: Patient Profile

Clinical: stage IV pancreatic cancer, prior treatment with FOLFIRINOX

Molecular: KRAS, TP53, ATM, KDM6A mutations; negative expression of TS, ERCC1, RRM1, TLE3; positive expression of TUBB3

1. Compile information from the electronic health report, including the type and stage of cancer, prior treatment information and other information about the patient including information on patient markers.

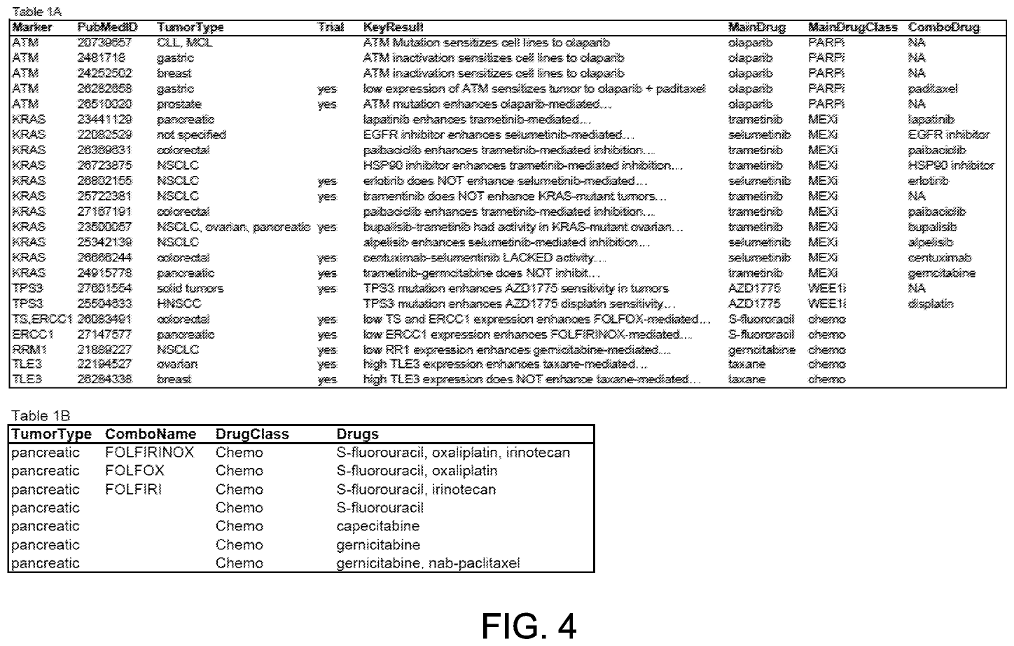

2. Query the knowledgebase to compile a list of relevant publications. Table 1A as included in FIG. 4 shows an illustrative list of publications that may be compiled here based on the patient's history and markers. Also, the database is queried to determine a list of standard-of-care options that match the patient's disease type as shown in Table 1B also included in FIG. 4. Other information used to query here may include disease stage or other demographic information related to the disease.

3. Remove any treatments that the patient has already received and had development of disease resistance and demographics. In this example, the patient did not progress on FOLFIRINOX, so it is left in as an option. In other cases, a treatment may not be appropriate based on the patient's age or demographic or other information in the patient's medical history.

4. Assign scores for the treatment options as shown below.

### Molecular Scores

The PARP inhibitor is given a score of 7 based on strong trial data. Others are given scores of 3 based on cell line or contradicting clinical evidence shown in the knowledgebase.

### Disease Relevance

Standard of care therapies for pancreatic cancer are given scores of 7.

PARP inhibitors are given a score of 6 based on promising evidence in pancreatic cancer.

MEK inhibitors are given a score of 4 based on less promising evidence.

WEE1 inhibitors are given a score of 3 based on still less promising evidence.

### Patient History

The patient has not had most of the options, therefore scores of 4 are assigned.

The patient has had FOLFIRINOX without progression, therefore a score of 2 is assigned.

Table 1C, reproduced below, shows the individual scores and the total score for each treatment option based on the patient's electronic health record, molecular databases and knowledgebase.

TABLE 1C

| Option | Molecular score | Disease score | Patient score | Total |
|---|---|---|---|---|
| PARP inhibitor | 7 | 6 | 4 | 17 |
| Gemcitabine- | 3 | 7 | 4 | 14 |
| based | | | | |
| 5FU-based | 3 | 7 | 2 | 12 |
| MEK inhibitor | 3 | 4 | 4 | 11 |
| WEE1 inhibitor | 3 | 3 | 4 | 10 |

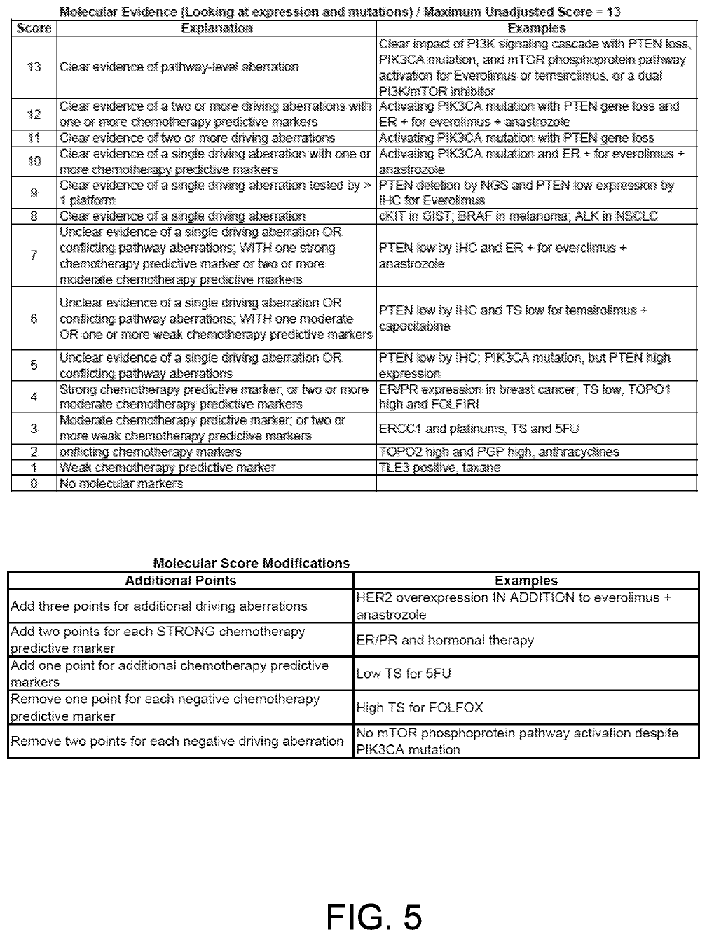

Scoring may be implemented in a variety of ways. According to one embodiment, a molecular score is given within a range of 0-13, a disease score is given within a range of 0-7 and a patient score is given within a range of 0-4. The scores are added to produce a total score and treatment options are ranked on the basis of the total score. It will be understood that changes may be made to those scoring ranges and different criteria may be used to set or adjust scores based on factors. Additionally, the individual scores may be added together to produce an overall score or the individual scores may be weighted prior to combining them to produce an overall score. A set of sample illustrative scoring rules for Molecular Evidence is shown in FIG. 5.

The different candidate treatments, scores, publications and other data associated with an individual disease case may be collected together an associated with the patient, typically via a patient identifier or other indicator of individual patients. This data associated with the patient may be referred to as a patient report. The patient report may include different formats and may be distributed to experts, physicians and/or patients in different formats and with different levels of interactivity, which allow experts or physicians to change scores, add notes, remove treatments and make other changes.

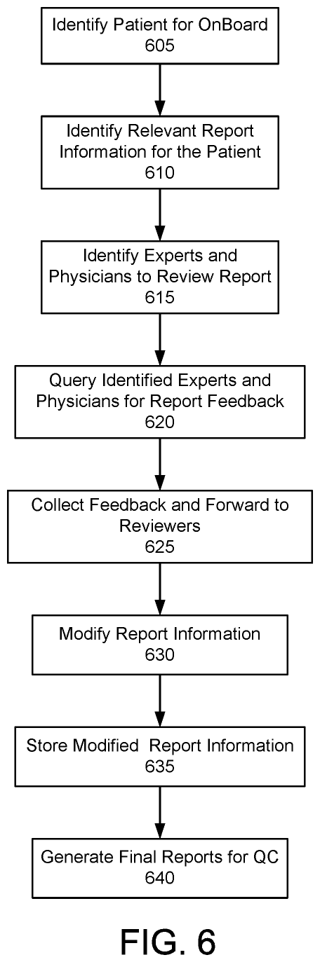

The patient report information or data may be made available to the OnBoard system shown in FIG. 1 to facilitate interactions among experts, physicians and others who are treating the patient, and quality control personnel, FIG. 6 depicts an illustrative process associated with the OnBoard system. A set of patients may be identified 605 as candidates for the OnBoard process. A management system for the OnBoard process may identify 610 relevant information for the patient and, based upon this information, identify 615 one or more experts and/or physicians to review the patient's history and report during the OnBoard process. The management system may query 620 the identified experts and physicians for their report feedback. The feedback may be collected 625 and forwarded to reviewers for additional analysis and critique.

Based upon the collected 625 feedback, the patient report may be modified 630. The modified report information may be stored 635 and a final report generated 640 for additional quality control review. The OnBoard process and system, as well as the report generation, is described in additional detail in the following discussion.

The OnBoard system may be implemented as a secure virtual disease or tumor review board (VTR) that provides expert review, facilitates dialogue between physicians, and enables collaboration on the specifics of a patient's case. In some embodiments, treating oncologists can actively participate in the tumor board process with top cancer specialists to review their patients' cases and recommend specific therapies. OnBoard is used by experts to review test results, analyze implications, identify and select the best treatment options and score them for each patient.

Upon login, users may access a case summary for each patient that provides information on patient medical history, molecular findings, and therapy options. In some implementations, OnBoard may have a multi-pane view that includes a chat box for clinicians to discuss the case, a flagging system to indicate high priority tasks, a list of medical review panel members working on the case and their approval status, and a panel to create and edit each part of a patient report.

OnBoard users may access molecular test results and patient progress notes, pathology, radiology reports and medical charts directly from OnBoard to analyze patient specific information and incorporate it into the report, Embedded access and quick links are provided to web portals including clinicaltrials.gov, Pubmed, NCI drug dictionary and other online resources.

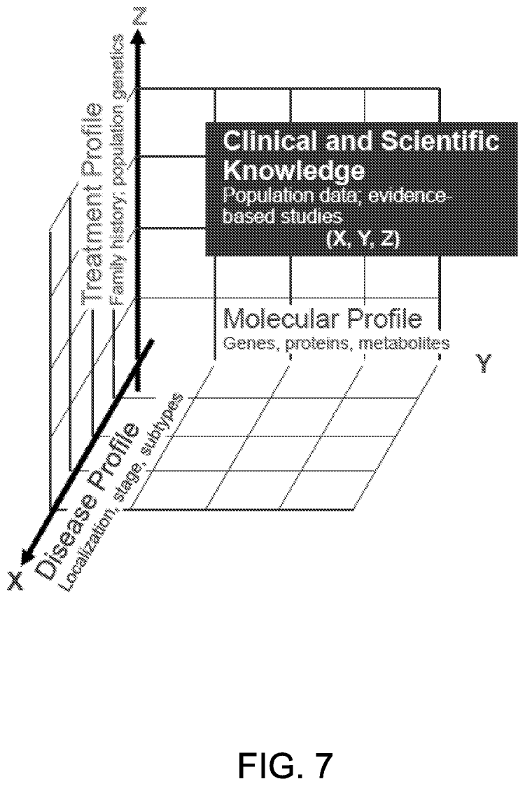

Users may also edit patient history, report narrative, therapy options, treatment considerations, relevant clinical trials, genomic and proteomic molecular profile, molecular implications, and drug implications. In the therapy options section of OnBoard, users can score therapies based on the molecular, disease and patient vectors in ExpO. A sample set of vectors can be seen in FIG. 7, wherein the disease profile vector is along the X-axis, the molecular profile vector is along the Y-axis, and the treatment profile vector is along the Z-axis. Users can create notes on each part of the patient report and make these notes private or share them with other members of the VTB. Users can also upload additional documents to make them a permanent part of the patient's case.

If a user has appropriate permissions, they can invite other doctors to participate in the VTB for a case. Once the VTB has agreed on the report recommendations they can generate a final report which is maintained as part of the patient's record, as well as being sent to the treating oncologist and patient.

A report undergoing or that has undergone the OnBoard process may be processed by a quality control system. This system may be an automated checker that checks data to verify that it is within ranges, that information after the OnBoard process remains accurate, and that the report formatting is correct, among other things. Quality control personnel may also be provided access to the report to perform control checking on the report. Once the report information is finalized, for example after undergoing a quality control process, the report information may be formatted into various formats for use, including a patient report, a physician report and a population level report. The contents of each report may be different. The physician reports may include additional information such as file notes and back and forth messaging between experts and the physician. The patient report may include the treatment options, scoring, information about the patient, disease, markers and publications on the treatment options and other information. The population level information may be anonymized as to each patient and instead collect information about treatments, diseases, markers and outcomes.

The systems shown in FIG. 1 may each comprise a server or other computer or mobile device that includes a processor that is coupled to a memory, input/output devices and network access technology. The memory stores computer program instructions that, when executed by the processor cause the processor to execute filtering, matching, database queries, scoring, messaging, analyzing, annotating and other specific processes shown and described herein. The input/output devices may include displays, keyboards, computer mice, touchscreens, speakers, microphones etc. The network access technology includes modems and other transmission and receiver technology to enable the processor to connect electrically, wirelessly or optically to computer networks such as LANs, WANs, or the Internet, or to connect to other computers, servers, mobile or other devices.

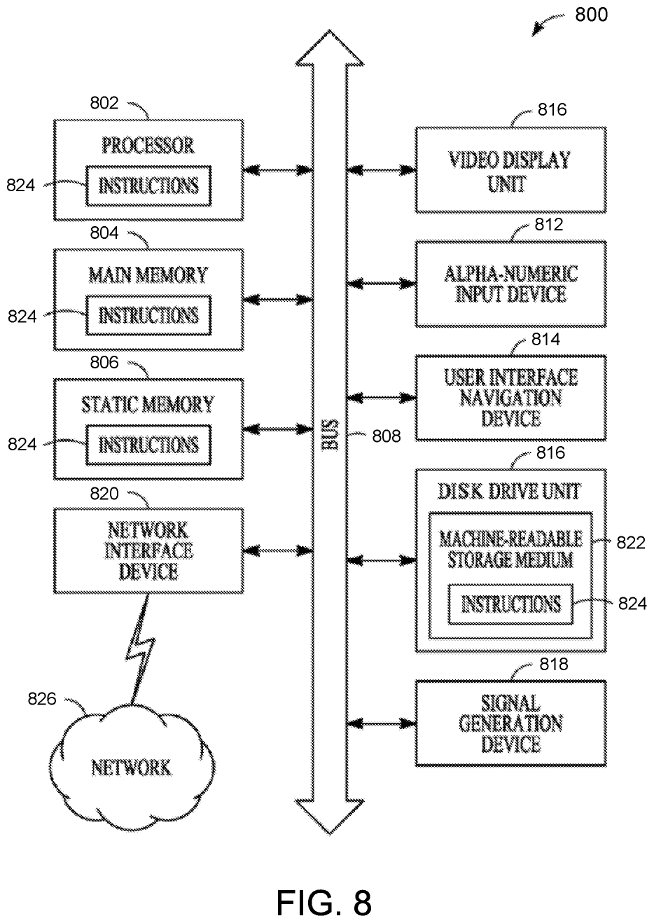

For example, FIG. 8 illustrates a block diagram that illustrates an example of a machine in the form of a computer system 800 within which instructions, for causing the computer system (e.g., one or more of the system devices as described above in discussion of FIG. 1) to perform any one or more of the methods discussed herein, may be executed. In various embodiments, the machine can operate as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a tablet PC, a set-top box (STB), a PDA, a cellular telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies and/or processes discussed herein.

The example computer system 800 includes a processor 802 (such as a central processing unit (CPU), a graphics processing unit (GPU), or both), a main memory 804 and a static memory 806, which communicate with each other via a bus 808. The computer system 800 may further include a video display unit 810 (such as a liquid crystal display (LCD) or a cathode ray tube (CRT)), an alpha-numeric input device 812 (such as a keyboard), a user interface (UI) navigation device (or cursor control device) 814 (such as a mouse), a disk drive unit 816, a signal generation device 818 (e.g., a speaker) and a network interface device 820.

The disk drive unit 816 includes a machine-readable storage medium 822 on which is stored one or more sets of instructions and data structures (e.g., software) 824 embodying or used by any one or more of the methods or functions described herein. The instructions 824 may also reside, completely or at least partially, within the main memory 804, static memory 806, and/or within the processor 802 during execution thereof by the computer system 800, the main memory 804, the static memory 806, and the processor 802 also constituting machine-readable media.

While the machine-readable storage medium 822 is shown in an example embodiment to be a single medium, the term “machine-readable storage medium” may include a single medium or multiple media (e.g., a centralized or distributed database, and/or associated caches and servers) that store the one or more instructions or data structures. The term “machine-readable storage medium” shall also be taken to include any tangible medium that is capable of storing, encoding or carrying instructions for execution by the machine and that cause the machine to perform any one or more of the methods of the present invention, or that is capable of storing, encoding or carrying data structures used by or associated with such instructions. The term “machine-readable storage medium” shall accordingly be taken to include, but not be limited to, solid-state memories, and optical and magnetic media. Specific examples of machine-readable media include non-volatile memory, including by way of example, semiconductor memory devices (e.g., erasable programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks. A “machine-readable storage medium” shall also include devices that may be interpreted as transitory, such as register memory, processor cache, and RAM, among others. The definition provided herein of machine-readable storage medium is applicable even if the machine-readable storage medium is further characterized as being “non-transitory.” For example, any addition of “non-transitory,” such as non-transitory machine-readable storage medium, is intended to continue to encompass register memory, processor cache and RAM, among other memory devices.

In various examples, the instructions 824 may further be transmitted or received over a communications network 826 using a transmission medium. The instructions 824 may be transmitted using the network interface device 820 and any one of a number of well-known transfer protocols (e.g., HTTP). Examples of communication networks include a LAN, a WAN, the Internet, mobile telephone networks, plain old telephone (POTS) networks, and wireless data networks (e.g., Wi-Fi and WiMAX networks). The term “transmission medium” shall be taken to include any intangible medium that is capable of storing, encoding or carrying instructions for execution by the machine, and includes digital or analog communications signals or other intangible media to facilitate communication of such software.

It should be noted that the computing system as shown in FIG. 8 is provided by way of example only. In practical applications, the processes as described herein can be implemented on a distributed computing system such as a cloud computing service having distributing processing and storage capabilities. For example, a cluster service such as the Elastic Container Service (ECS) in combination with the Simple Storage Service (S3), both provided by Amazon Web Services, can be used to provide a cloud-based implementation system.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the claims include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method of generating a therapy plan for a patient, the method comprising:
providing a knowledgebase of annotated disease treatment information, a patient history database including disease types for a plurality of patients and a molecular database including molecular data associated with patients;
selecting at least one of the plurality of patients for analysis;
identifying a list of one or more candidate therapy plans from the knowledgebase based on at least one of the disease type and the molecular data for the at least one patient; and
scoring each of the one or more candidate therapy plans by combining a molecular vector, a disease-specific vector and a patient history vector.

2. The method of claim 1, wherein:
the molecular database comprises information concerning genomic and protein profiles of the at least one patient;
the knowledgebase comprises information concerning a disease in the at least one patient; and
the patient history database comprises information concerning a prior therapy history of the at least one patient.

3. The method of claim 1, wherein:
the molecular vector is determined according to scoring rules based on the molecular database;
the disease-specific vector is determined according to scoring rules based on the knowledgebase; and
the patient history vector is determined according to scoring rules based on the patient history database.

4. The method of claim 1, wherein the identifying the list comprises filtering initial therapy plans based on the patient history database.

5. The method of claim 1, wherein the identifying the list comprises identifying a disease or a state of disease progression in the at least one patient based on the molecular database, the knowledgebase, and the patient history database.

6. The method of claim 1, wherein the identifying the list comprises receiving at least one of (i) a query result from the molecular database concerning the molecular data of the at least one patient, (ii) a query result from the knowledgebase concerning the disease type of the at least one patient, and (iii) a query result from the patient history database concerning a prior therapy history of the at least one patient.

7. The method of claim 1, wherein the scoring comprises ranking the list of one of more candidate therapy plans.

8. The method of claim 1, wherein the therapy plan comprises a pharmaceutical treatment plan with one or more pharmaceutical drugs.

9. The method of claim 1, wherein the at least one patient is a cancer patient.

10. The method of claim 1, wherein the at least one patient is a pancreatic cancer patient.

11. The method of claim 1, wherein the molecular data includes genomic and protein profiles of the at least one patient.

12. A method of treating cancer, the method comprising:
generating a therapy plan for a cancer patient according to the method of any one of the proceeding claims, and
administering one or more pharmaceutical drug in accordance with the therapy plan.

13. A system for generating a therapy plan for a patient, comprising:
a knowledgebase of annotated disease treatment information, a patient history database including disease types for a plurality of patients, and a molecular database including molecular data associated with patients;
a memory including program instructions for assembling molecular, disease-specific and patient history vectors from the databases, and filtering and scoring at least one candidate therapy for at least one of the plurality of patients; and
a processor coupled to the databases and the memory capable of executing the program instructions to:
identify a list of one or more candidate therapy plans from the knowledgebase based on at least one of the disease type and the molecular data for the at least one patient,
compile a molecular vector, a disease-specific vector and a patient history vector, and
score each of the at least one candidate therapy plans for each of the at least one patient based on the molecular vector, the disease-specific vector and the patient history vector.

14. The system of claim 13, wherein:
the molecular database comprises information concerning genomic and protein profiles of the at least one patient;
the knowledgebase comprises information concerning a disease in the at least one patient; and
the patient history database comprises information concerning a prior therapy history of the at least one patient.

15. The system of claim 13, wherein:
the molecular vector is determined according to scoring rules based on the molecular database;
the disease-specific vector is determined according to scoring rules based on the knowledgebase; and
the patient history vector is determined according to scoring rules based on the patient history database.

16. The system of claim 13, wherein the identifying the list comprises filtering initial therapy plans based on the patient history database.

17. The system of claim 13, wherein the identifying the list comprises identifying a disease or a state of disease progression in the at least one patient based on the molecular database, the knowledgebase, and the patient history database.

18. The system of claim 13, wherein the identifying the list comprises receiving at least one of (i) a query result from the molecular database concerning the molecular data of the at least one patient, (ii) a query result from the knowledgebase concerning in the disease type of the at least one patient, and (iii) a query result from the patient history database concerning a prior therapy history of the at least one patient.

19. The system of claim 13, wherein the scoring comprises ranking the list of one of more candidate therapy plans.

20. The system of claim 13, wherein the therapy plan comprises a pharmaceutical treatment plan with one or more pharmaceutical drugs.

21. The system of claim 13, wherein the at least one patient is a cancer patient.

22. The system of claim 13, wherein the at least one patient is a pancreatic cancer patient.

23. The system of claim 13, wherein the molecular data includes genomic and protein profiles of the at least one patient.
